(19) United States
(12) Patent Application Publication
(10) Pub. No.: US 2024/0197246 A1
(43) Pub. Date: Jun. 20, 2024

(54) Classifying User Activity Using Multiple Sensors

(71) Applicant: Samsung Electronics Co., Ltd., Suwon-si (KR)

(72) Inventors: Ebrahim Nematihosseinabadi, San Jose, CA (US); Mohsin Ahmed, Sunnyvale, CA (US); Nafiul Rashid, Irvine, CA (US); Jilong Kuang, San Jose, CA (US); Jun Gao, Menlo Park, CA (US)

(21) Appl. No.: 18/118,528

(22) Filed: Mar. 7, 2023

Related U.S. Application Data
(60) Provisional application No. 63/433,721, filed on Dec. 19, 2022.

(51) Int. Cl. A61B 5/00; A61B 5/11

(52) U.S. Cl. CPC A61B 5/6802; A61B 5/1118; A61B 5/7264

(57) ABSTRACT

In one embodiment, a method includes accessing first sensor data from a first sensor worn on a first portion of a user's body and accessing second sensor data from a second sensor worn on a second portion of the user's body. The method includes determining, based on both the first sensor data and the second sensor data, one or more first features related to the user's activity and determining, based on the first features, an initial classification of the user's activity. When the initial classification indicates a class that includes one or more subclasses that are more distinguishable by one of the sensors, then a specific subclassification may be determined based on sensor data from only that one sensor. Otherwise, the classification of the user's activity may be based on the one or more first features that use data from both sensors.

### PRIORITY CLAIM

This application claims the benefit under 35 U.S.C. § 119 of U.S. Provisional Patent Application No. 63/433,721 filed Dec. 19, 2022, and incorporated by reference herein.

### TECHNICAL FIELD

This application generally relates to classifying user activity using multiple sensors.

### BACKGROUND

Various sensors may be incorporated into consumer electronic devices, including devices that are worn on a user's body. For example, sensor electronics may be included in devices such as a wrist-worn watch, a ring, an activity-tracker, a necklace, clothing, and/or head-worn devices such as headphones, earbuds, headbands, etc. Sensors embedded in such devices can include sensors that detect motion, position, EM waves, audio or other mechanical waves, and so on. For example, a microphone may be embedded in a user-worn device, such as in a watch. As another example, an inertial measurement unit may be embedded in a user-worn device, such as in headphones or earbuds.

### BRIEF DESCRIPTION OF THE DRAWINGS

FIG. 1 illustrates an example of device positions given user positions.

FIG. 2 illustrates an example method for using sensor data from two or more sensors to determine a user's activity.

FIG. 3 illustrates an example architecture for performing the method of FIG. 2.

FIG. 4 illustrates example accelerometer sensor data.

FIG. 5A illustrates an example accelerometer signal from a pair of earbuds.

FIG. 5B illustrates an example of chunk and bubble data processing.

FIG. 6 illustrates an example computing system.

### DESCRIPTION OF EXAMPLE EMBODIMENTS

Sensors may be used to track the motion or orientation of a user's body or a portion of the user's body. For example, RGB cameras in an environment of a user may be used to capture images of a user to detect the user's motion and/or posture. However, multiple cameras typically need to be placed in the user's environment and the user must be in the camera's field of view, which is expensive and raises privacy concerns. As another example, RF-ID based or Wi-Fi based sensing may be used based on signals reflecting off a user's body, but such sensing methods are inaccurate, prone to noise, and require the equipment to be within the environment of the user, which is impractical for many user locations (e.g., outdoors). Sensors may be physically attached to multiple portions of a user's body, but wearing numerous sensors may be uncomfortable and burdensome for a user.

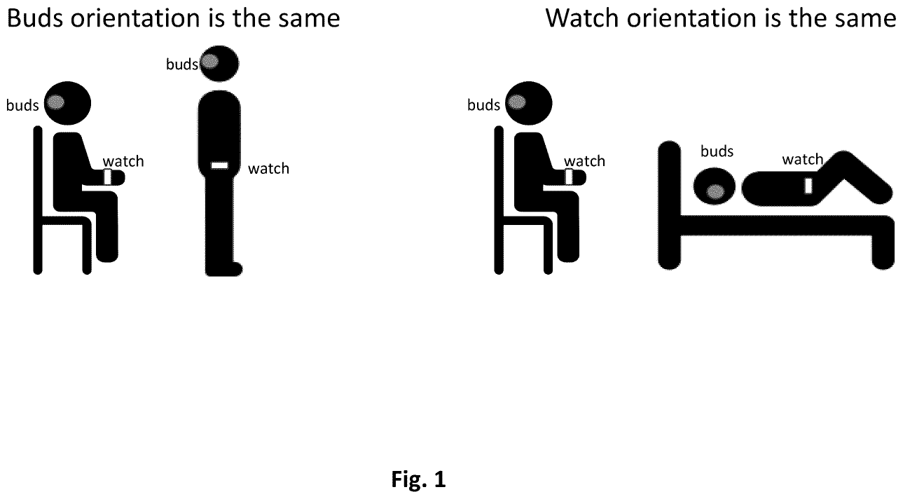

Some of these challenges may be overcome by incorporating sensors in body-worn user devices that serve other purposes, which increase user desire to wear the devices. For example, sensors may be incorporated in wrist-worn devices, such as a watch, or in head-worn devices, such as headphones. Incorporating sensors in such devices may make wearing sensors much more convenient for a user. However, inaccuracies may occur when using such sensors to detect user motion or position. For example, for an arm-worn (e.g., wrist-worn) sensor, hand position or motion does not reflect body position or movement. For example, FIG. 1 illustrates an example in which a watch is in the same spatial orientation when a user is sitting or lying down, and thus an orientation sensor in the watch is unable to accurately determine the user's orientation (e.g., sitting vs lying down). Likewise, FIG. 1 illustrates that an orientation sensor in a head-worn device (e.g., earbuds) may not be able to distinguish between, e.g., sitting and standing, as the user's head may be in the same orientation in both circumstances. Such body-worn devices may also suffer inaccuracies when detecting motion. For instance, a wrist-worn device may have high within-class variance due to the similar attributes regarding wrist movements; for example, while a person is in a stationary posture, any hand motion could disturb posture detection models. In addition, portions of the arm such as the wrist are far from the body's center of mass, which can cause relatively low accuracy for posture detection and activity recognition. As another example, a large number of activities of daily living involves hand motion that cannot be captured by a device worn elsewhere, such as on the head. For example, motions related to eating, drinking, brushing teeth, etc. may all share similar head positions and/or have limited head movement.

Combining, or fusing, the data from multiple sensors can solve some of the problems mentioned above. However, fusing data can also create issues with detecting a user's motion and/or orientation. For example, late (e.g., decision) fusion can lead to a loss of useful information obtained by combining data from multiple sensors prior to decision, such as a motion or orientation classification. On the other hand, early or intermediate fusion can give undue weight to data from each sensor, for instance because in many circumstances (such as shown in FIG. 1), data from a particular sensor may be more informative than data from other sensors.

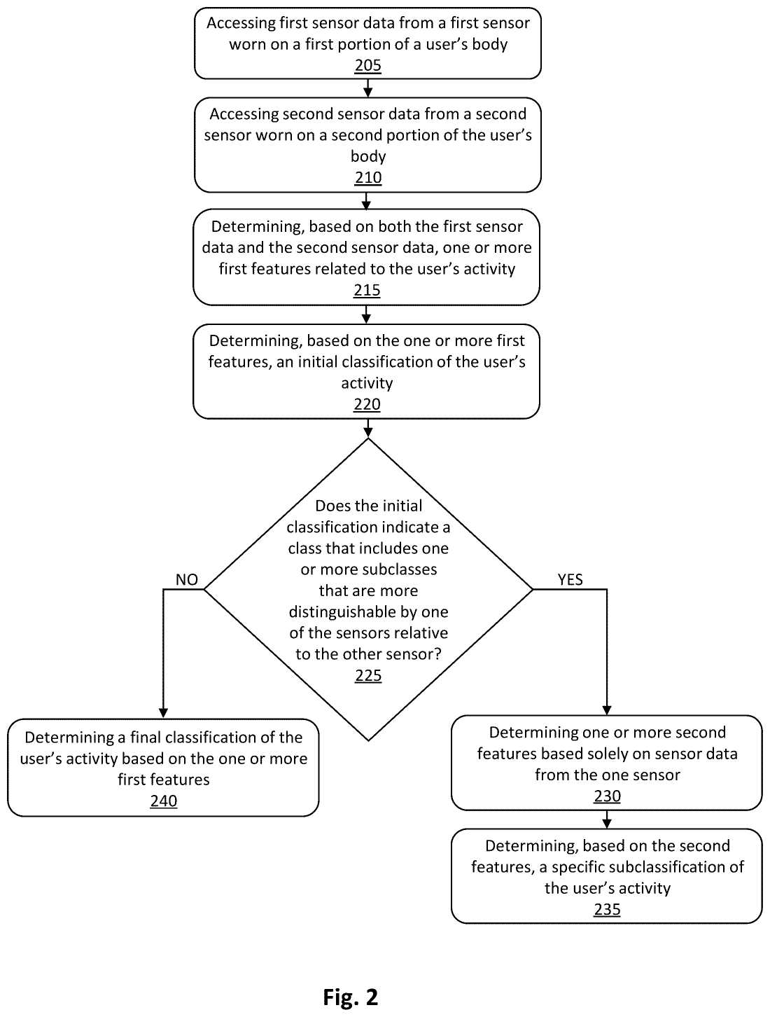

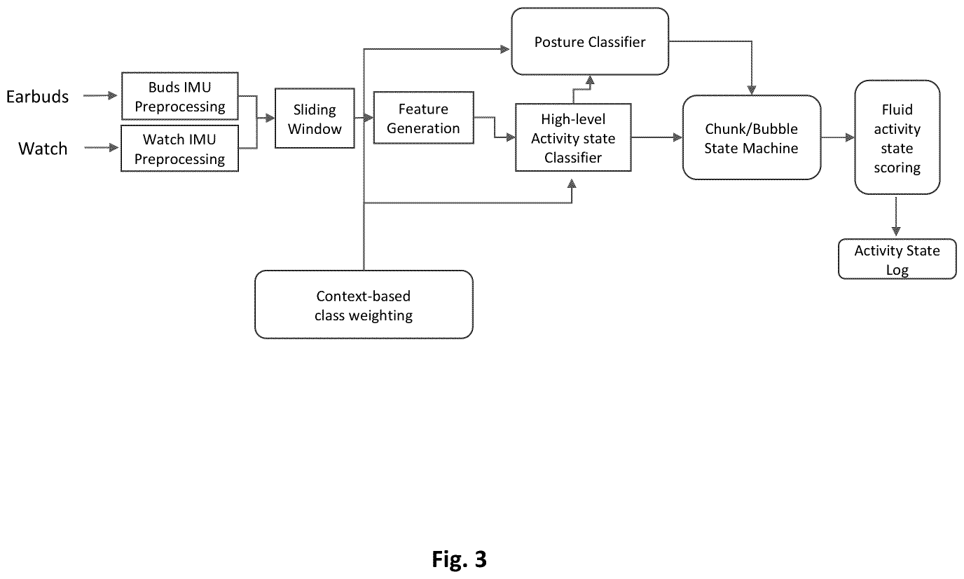

FIG. 2 illustrates an example method for using sensor data from two or more sensors to determine a user's activity. FIG. 3 illustrates an example architecture for performing the method of FIG. 2, along with certain other functionality described herein. In addition to solving the problems discussed above, particular embodiments described herein improve detection and classification accuracy by correcting short-term inaccuracies that occur due to similarities between classes. For example, a seated user might use her hand to write something or change the channel of a TV. In this situation the motion sensor signal from the hand could look similar to other activities such as walking, making accurate classification difficult.

Step 205 of the example method of FIG. 2 includes accessing first sensor data from a first sensor worn on a first portion of a user's body, and step 210 of the example method of FIG. 2 includes accessing second sensor data from a second sensor worn on a second portion of the user's body. As explained above, the first sensor may be integrated within a watch (or other wrist-worn device, such as an activity tracker, etc.) and the second sensor may be integrated within a headset, such as earbuds, but this disclosure contemplates other arrangements as well. For example, the first sensor may be located on a limb, such as a leg or arm, of the user, while a second sensor may be located on the user's trunk (e.g., integrated in a shirt or other clothing, integrated in a necklace, etc.) or located on the user's head, such as a headband, pair of glasses, a face covering such as a mask, etc.

In particular embodiments, the accessing of step 205 or step 210 (or both) may include capturing the sensor data. In particular embodiments, one or more of those steps may include accessing the sensor data from the device in which the sensor is integrated (e.g., from the watch or headset in which the sensor is integrated), and/or may include accessing such data from an intermediary client or a server computing device. For example, a smartwatch may be paired with or otherwise connected to a user's smartphone, tablet, computer, or server device, and the smartwatch may transmit sensor data to such devices, from which the sensor data may be accessed. In particular embodiment, steps 205 and/or 210 may include receiving the sensor data from the respective devices in which the sensors are integrated or from another electronic device, such as an intermediary electronic device that has received the data. In particular embodiments, the steps of the example method of FIG. 2 may be performed by a single device, such as by one of the devices in which the sensors are integrated, or by another computing device such as a smartphone, computer, server device, etc. In particular embodiments, the steps of the example method of FIG. 2 may be performed by more than one computing device. For example, a smartphone may access sensor data and extract corresponding features, while another device (e.g., a computer, a server, etc.) may perform some or all of the classification described in connection with the example method of FIG. 2.

Step 215 of the example method of FIG. 2 includes determining, based on both the first sensor data and the second sensor data, one or more first features related to the user's activity. For example, the first sensor data and the second data may be temporally linked, or fused, and the linked data may be used to determine activity-related features associated with the linked sensor data. FIG. 3 illustrates a specific embodiment of an architecture that performs step 215 of the example method of FIG. 2. In the example of FIG. 3, the first sensor may be part of a watch and the second sensor may be part of a headset, such as a pair of earbuds. Each sensor may be a sensor that detects signals related to position and movement, such as a 6-axis inertial measurement unit. As illustrated in the example of FIG. 3, sensor data may undergo preprocessing. For example, the sensor data may be smoothed, passed through a high-pass filter, and normalized. As illustrated in the example of FIG. 3, a sliding window may be used to generate linked chunks of IMU data from the watch IMU and the earbuds IMU, such that the IMU data from each device is temporally linked. Features may then be generated from the linked (or fused) IMU data chunks from the two different sensors.

The generated features may be used to perform an initial classification of the user's activity. For example, as illustrated in FIG. 3, a high-level activity state classifier may perform an initial classification of the features generated from the fused sensor data. As illustrated in FIG. 2, step 220 of the example method of FIG. 2 includes determining, based on the one or more first features, an initial classification of the user's activity. The initial classification may include an initial, higher-level set of class labels that broadly describe the user's activity as coarse level classifier. For example, initial class labels may include a more stationary class such as the posture label (which may include posture-based activities such as sitting, standing, lying down, etc.), and a more non-stationary class such as moving label (which may include any activity that has both body motion and a location change, e.g., a location change of the user's center of mass), and a non-moving activity label (which may include any activity that has body motion but not location change, e.g., doing pushups, brushing teeth, etc.). This disclosure contemplates that other initial classification labels may be used alternatively, or in addition to, the example initial class labels described above.

As illustrated in FIGS. 2 and 3 and explained herein, multiple sensors may provide superior detection capabilities for user activities compared to using only one device. For example, using data from sensors on two different devices provides more robustness against noise. Moreover, in particular embodiments, one sensor (e.g., in a watch) is capable of accurately capturing hand movements while another sensors (e.g., in a device on the user's trunk or head) may accurately capture motion of the user's center of mass, and combining these motion profiles can more accurately detect and identify many human motions that are characterized by both hand movements and center-of-mass movement (e.g., running or walking), or lack thereof (e.g., lying down). This disclosure contemplates that sensors worn on additional or other body locations (e.g., on the user's leg) may also be used to capture limb movement and center-of-mass movement, and as described throughout, this disclosure is not limited to sensors worn on the wrist and on the head.

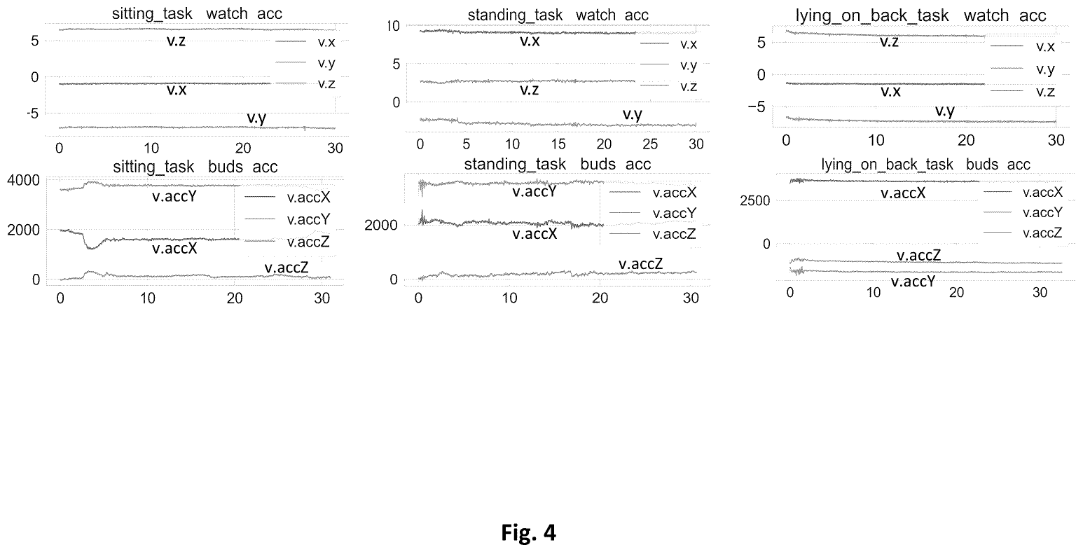

As illustrated in FIGS. 2 and 3, sensor data is fused (e.g., from sensors worn on the user's wrist and on the user's head), and classifying activities based on features determined from this fused sensor data may improve the system's ability to detect many kinds of human motions and improve the system's robustness to noise. However, as explained above, sensors in some locations may poorly differentiate between specific kinds of human activities. For example, FIG. 4 illustrates example accelerometer sensor data from a sensor in a watch and from a sensor in earbuds for user activities that including sitting down, standing, and lying down. As illustrated in FIG. 4, a watch worn on a user's wrist generates accelerometer data that is very similar when a user is sitting or lying down, while in contrast, the watch's accelerometer data when the user is standing is different from the data that is generated when the user is sitting or lying down. Likewise, as illustrated in FIG. 4, a set of earbuds worn on a user's head generates accelerometer data that is very similar when a user is sitting or standing, while in contrast, the earbuds's accelerometer data when the user is lying down is different from the data that is generated when the user is standing or sitting. As explained herein, this can be generalized for other types of activities and for various types of sensors and placement of them.

Step 225 of the example method of FIG. 2 includes determining whether the initial classification indicates a class that includes one or more subclasses that are more distinguishable by one of the sensors relative to the other sensor. If the answer is determined to be yes, then step 230 of the example method of FIG. 2 includes determining one or more second features based solely on sensor data from the one sensor that is more able to distinguish the appropriate subclass(es) than the other sensor. FIG. 3 illustrates an example of steps 225 and 230. If the high-level activity state classifier in FIG. 3 classifies the user activity as a posture based on features generated from the fused sensor data, then a posture classifier is used to detect the specific posture subclass (e.g., sitting, standing, lying down, etc.). For example, if “posture” is the initial classification, then data from the watch's sensor may be initially examined (either the preprocessed sensor data or features generated from that sensor data). If the data (either as preprocessed, or using the resulting features) from the watch's sensor by itself (i.e., not fused with the earbud sensor data) indicates the specific subclassification, then the specific subclassification is selected as the classification of the user's activity. This corresponds to step 235 of the example method of FIG. 2, which includes determining, based on the second features, a specific subclassification of the user's activity. If the data from the watch's sensor by itself does not distinguish among or between various subclasses (e.g., the sensor data ambiguously identifies “sit” or “lying down” subclasses), then the data (either as preprocessed, or using the resulting features) obtained from the earbuds sensor is by itself examined (i.e., not fused with the watch sensor data) to determine the specific subclassification, and that specific subclassification is selected as the classification of the user's activity

The example above describes specific sensors, classifications, and subclassifications, but this disclosure contemplates any suitable sensor combinations, classes and classifications, and subclasses and subclassifications. Moreover, while the example above describes two sensors, this disclosure contemplates that three or more sensors in three or more devices may be used, and in such embodiments, subclassifications may be made by more than one sensor but less than all sensors, if at least one of the sensors is unable to distinguish between various subclasses.

As illustrated in step 240 of FIG. 2 and in FIG. 3, if the determination in step 225 is “no,” i.e., the initial classification does not indicate a class that includes one or more subclasses that are more distinguishable by one of the sensors relative to the other sensor, then the final classification is determined based on the first features. In particular embodiments, the final classification may be the initial classification, e.g., the high-level classifier in FIG. 3 may determine the specific final classification for the user's activity. In particular embodiments, a separate subclassifier may specify, using the features determined from the fused sensor data, the specific subclassification for the user's activity. In particular embodiments, step 240 may be determined at the same time as step 220.

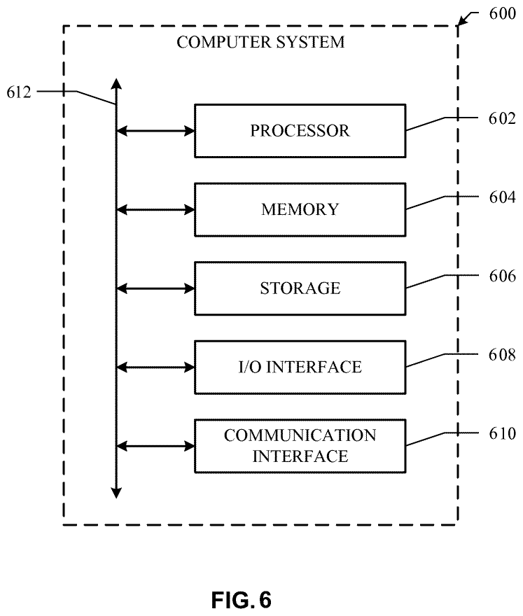

Particular embodiments may repeat one or more steps of the method of FIG. 2, where appropriate. Although this disclosure describes and illustrates particular steps of the method of FIG. 2 as occurring in a particular order, this disclosure contemplates any suitable steps of the method of FIG. 2 occurring in any suitable order. Moreover, although this disclosure describes and illustrates particular components, devices, or systems carrying out particular steps of the method of FIG. 2, such as the computer system of FIG. 6, this disclosure contemplates any suitable combination of any suitable components, devices, or systems carrying out any suitable steps of the method of FIG. 2. Moreover, this disclosure contemplates that some or all of the computing operations described herein, including the steps of the example method illustrated in FIG. 2, may be performed by circuitry of a computing device, for example the computing device of FIG. 6, by a processor coupled to non-transitory computer readable storage media, or any suitable combination thereof.

When logging a user's activity states, particular embodiments continuously track the posture and activity state of the user over a relatively long period of time to generate a log history. Such embodiments should be insensitive to sudden short-duration movements of the sensor devices. For example, a seated user might use her hand to write something or change the channel of a TV. In this situation a sensor signal from a device worn near the hand could look similar to sensor signals generated during other activities, such as walking, and therefore misclassification of the user's activity might happen. Particular embodiments might attempt to address this misclassification during training by including hand-movement sensor signals while the user is seated as all part of a “sit” class to resolve the issue at the observation level. However, this could lead to class pollution as data from various classes could look interchangeably similar, e.g., similar hand motions made during walking may be misclassified as being associated with “sitting.” In addition, training even one class for many different types of user gestures or movements can be a very burdensome data collection process.

Particular embodiments may instead address such misclassification by using a state machine that is applied to the signal in the decision level. Instead of attempting to correctly label all data during the classification phase (e.g., labelling various kinds of hand motions made while sitting as a “sit” activity), embodiments using the state machine described in this section let the misclassification occur. The state machine then corrects the misclassification in the decision layer.

FIG. 3 illustrates an example chunk/bubble state machine that receives classification data. As used here, a “chunk” represents a certain amount (or chunk) of consecutive predictions from one specific class, or state, and “bubble” represents data labelled as any different class that sits in between two chunks. Particular embodiments identify chunks and bubbles in the data output from the prediction/classification layer and then convert bubble classification labels to the surrounding chunk labels if conversion criteria are met.

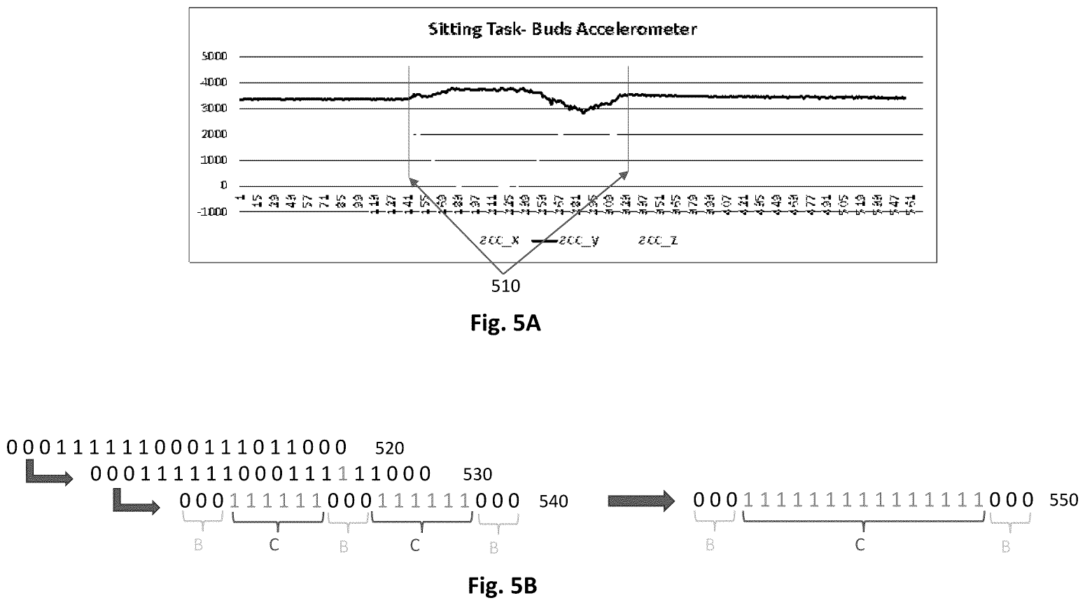

For example, FIG. 5A illustrates an example accelerometer signal from a pair of earbuds where the user is sitting straight with no motion but then suddenly tilts her head to the right side and then moves her head back to the upright normal position. The sensor data during head movement is illustrated as portion 510, and portion 510 may result in classification of the user's activity during the time associated with data 510 as something other than “sit” (e.g., as “running). As illustrated in FIG. 5A, the accelerometer sensor data surrounding portion 510 is identified in this example to the “sit” class.

In particular embodiments the state machine includes at least three main modules. The first module may be a prediction-smoothing filter that applies an initial smoothing algorithm to remove very short changes in the classification data received by the state machine. As a result, for example, one isolated “sit” label between two “stand” labels is automatically converted to “stand”. This is different from bubble label conversion, which is described below. FIG. 5B illustrates an example of this functionality. Label (classified) stream 520 illustrates the input classification data received by the state machine from the classification layer. In the example of FIG. 5B, label stream 520 includes two output predicted classes, e.g., a class identified as “0” and a class identified as “1.” In particular embodiments, the prediction-smoothing filter operates on label stream 520 and outputs label stream 530 by changing the isolated “0” label (i.e., the single “0” label surrounded by other class predictions) to a “1” label.

A second module included in particular embodiments of the state machine is a chunk/bubble divider that divides the classified data into chunks and bubbles. For example, a portion of classified data may be identified as a chunk if there are at least n consecutive predictions from the same label, such that n is a threshold for determining whether consecutive data sharing a class label is identified as a chunk. As another example, a portion of classified data may be identified as a chunk if there are least x nearby predictions from the same label, even if some of those x predictions are separated by an interruption (i.e., data with another class label) of maximum length of y (where x >>y). In particular embodiments, n, x, and/or y may be determined empirically during model training. In particular embodiments, all groups of classified data not labeled as a “chunk” are then labelled as a “bubble.” For example, labelled stream 540 illustrates stream 530 with the classified data identified as a “chunk” or as a “bubble.”

A third module included in particular embodiments of the state machine is a bubble re-labeler. For example, any sufficiently small bubble (e.g., a bubble of less than a threshold size k, for example as estimated during training) that is surrounded by two chunks having the same label may have that bubble's label reassigned to the label of the surrounding chunks. For example, as shown in data stream 550 FIG. 5B, a bubble B sitting between two chunks C has had its class label changed to “1,” which is the class label of the two surrounding chunks C. If relabeling criteria are not met, then in particular embodiments a small bubble may be labelled as “unknown,” i.e., its initial class label from the classification layer is removed and it is not assigned another classification label. In particular embodiments, any bubble that is not identified as sufficiently small may not be relabeled. This design may be extended to various types of chunks (chunk type1, chunk type 2, etc) and various types of bubbles (bubble type1, bubble type2, etc.) with various sets of conditions for each type based on the types of the activities to be logged.

In particular embodiments, a system as described herein may encounter situations in which data collected by a sensor is too noisy or otherwise unreliable to be useful for determining a user's activities. As described below, particular embodiments identify and avoid processing such data, increasing the accuracy when identifying user activities and conserving computational resources that would otherwise be devoted to processing unreliable data (e.g., preserving the battery life of a smartphone that processes sensor data).

For example, data may be collected by a sensor in environments that contains high levels of motion noise. Particular embodiments may continuously measure the motion noise of a sensor to identify low-quality data segments, for example by using signal intensity and/or standard deviation to determine whether the sensor data is above a data threshold, signaling an unusually highly active IMU episode. If yes, then the corresponding data segments can be discarded without wasting computational resources on those segments or having those segments corrupt activity identification log.

As another example, if a sensor is in a device that is not worn properly, then a decrease or change in sensor data usually results in misclassification. For example, earbuds may not be worn properly, e.g., if they are not properly placed in the ear, or sit on a table rather than being worn, etc. particular embodiments may identify episodes where earbuds (or other device, as the use case may be) are “not worn” or “worn improperly” and drop the corresponding data, for example based on sensor-data signatures associated with the “not worn” or “improperly worn” statuses. Particular embodiments may apply this criteria independently to the left or right earbud, and particular embodiments may also select a left or right earbud to use for sensor data based on, for example, a battery level of the respective earbuds. As another example, a sensor may be part of a watch that is worn loosely or worn on the wrong hand, which may result in decreased sensor accuracy. Particular embodiments may implement a binary classifier (e.g., based on IMU data from the watch) to identify how the watch is worn (e.g., tight or loose). As explained above, if a data segment from a sensor is determined to be unreliable then that data segment may be discarded; otherwise, the data segment may be sent for data preprocessing, e.g., as described in connection with the example of FIG. 3.

Particular embodiments, for example as illustrated in FIG. 3, ultimately output an activity-state log that describes the amount of time over a period of time (e.g., an hour, a day, a week, etc.) that a user has engaged in certain activities, such as running, walking, lying down, sleeping, sitting, etc. In such embodiments, it may be more important to accurately identify a user's activity over this period of time rather than accurately detecting the exact activity performed at each specific instance within that period of time. Moreover, as described herein, detections of activity states at any particular instant in time may be erroneous for certain activities when a sensor is providing unreliable data, and in two-device, two-sensor embodiments, this means that classification is made by only a single sensor. However, as explained above a single sensor may not be able to distinguish between activities in some scenarios; for example, sensor data associated with a set of earbuds may look very similar for sitting and standing activities. If the actual state is sitting but a classifier receiving the earbuds sensor data results in relatively higher probability for the “standing” class, then in conventional techniques the activity in this instant is labelled only as “standing,” and these kinds of errors can grow and accumulate over the period of time used in the activity log.

Instead of assigning a set of data (e.g., 1 second worth of data) a class label using only the class with the highest probability as determined by the classification layer, particular embodiments of this disclosure use a fluid scoring system where that set of data (e.g., 1 second) may be assigned to multiple different classes. In particular embodiments, this assignment may be based on the probability of each of the detected class as well as the rate of misclassification in training. As one example, in a two-device system including a watch and a set of earbuds, if the watch is detected as not being worn during a period of time, then activity logging for that period of time will be based on data only from one or more sensors in the earbuds. In this example, if any of the sit or stand class is detected during the time period that only earbud data is used to log user activity, then sit and stand may both be logged during this time period because the earbuds are incapable of correctly differentiating the two classes without data from the sensor in the watch (as explained before), for example based on their respective probabilities of matching the actual user activity.

In particular embodiments, during system training data may be obtained from only one device in a two-device system (e.g., from only a watch, or from only earbuds) for each of a set of classes (e.g., sit, stand, lie), and the false negatives FN may be measured for each class during a particular user activity. For example, using only a watch and with the user in a known sitting state, the number of instances (or false negatives) in which the user's state is labeled as “standing” or “lying down” may be measured. The same process may occur for each state, and may occur again using only the earbuds. Then, if only one device is used during data collection and user activity logging, activity states may be logged based on both the probability of that state, as determined by the classification layer in that given instance, and the number of false negatives for that state/device combination determined during training. For example, in a three-state system containing states x, y, and z, a user's activity during a particular time period may be logged as corresponding to state x according to log, =(Pₓ+F̅N̅_{x,y}+F̅N̅_{x,z})_{normalized}, where Pₓ is the probability of state x as determined by the classification layer and F̅N̅_{a,b} is FNₐ in state b divided by the true positives in state b normalized using an a factor, where a is determined empirically during training. The user's activity during that particular time period may be logged as corresponding to states y and z in the same manner (e.g., log_{y}=(P_{y}+F̅N̅_{y,x}+F̅N̅_{y,z})_{normalized} and log_{z}=(P_{z}+F̅N̅_{z,x}+F̅N̅_{z,y})_{normalized}). For example, if data is classified every second, then 0.4 seconds may be attributed to sitting down, 0.4 to lying down, and 0.2 to standing. This distribution may be reflected in the user's activity log, which identifies the amount of time the user spent in various states over a period of time (e.g., an hour, day, week, etc.). In particular embodiments, and as indicated in some of the examples above, this type of fluid activity logging may be used only when a sensor or group of sensors collects data that ambiguously corresponds to multiple classes (e.g., when only a watch is used and the data reflects that the user is either sitting down or lying down).

Particular embodiments may use both data from sensors and contextual information to classify a user's activity. In particular embodiments, the contextual information may be obtained by the same devices in which the sensors are located. In particular embodiments, information from other devices may also or alternatively be used to determine contextual information relevant to activity classification. As one example, contextual information may include a time of day. For instance, if data is obtained during nighttime, the classification layer could give more weight to a “lying down” or “sleeping” class compared to other activity classifications. As another example, contextual information may include a location status. For example, an outdoor vs. indoor status may be used, and when the user is indoors, the classification layer may give more weight to “stand” and “sit” classes, while when outdoors, the classification layer may give more weight to “move” than to “non-move” categories. As another example, contextual information may include a user's age. For instance, if a user is older than a certain age, more weight may be given to non-moving classes vs. moving classes. Examples of other contexts include, but are not limited to, periodicity, activity state in past x minutes, etc.

FIG. 3 illustrates an example embodiment in which contextual information may be used to weight particular classes in the classification layer. In particular embodiments, cost-sensitive classification can be a fit solution for including contextual information in classification decisions. For example, each class may receive a weight w, based on cost values in a cost matrix generated from the contextual information, for example:

$$w ( i ) = \frac{nc ( i )}{{\sum }_{k = 1}^{κ} n ( k ) c ( k )}$$

where c(i) is the sum of the iₜₕ column of the cost matrix such that:

$$c ( i ) = {\sum }_{j = 1}^{κ} c ( j , i )$$

where i and j represent the row and column position in the cost matrix. Based on the cost matrix, a class that has a relatively higher cost will receive less weight than a class with a relatively lower cost when generating prediction probabilities. The cost matrix can be generated using expert input, by collecting in-home data and labels, and/or during training. For example, an expert may conclude that more cost should be given to a “lie down” class when a user is outdoors during the day.

As discussed above, particular embodiments of this disclosure are directed to providing a log, e.g., a histogram, of user activity over a period of time rather than on instantaneous activity labelling. In other words, a primary purpose of particular (but not necessarily all) embodiments is to provide the user with an accurate synopsis of activities over time rather than with an instantaneous classification. Particular embodiments may include “unknown” as a prediction class when a confidence of classifier is relatively low, and only log an activity when the classifier confidence is relatively high.

Particular embodiments may enable longitudinal monitoring of logged activities and may identify common patterns for a particular user. For example, particular embodiments may access a user log and, for various classes, extract both temporal and non-temporal features. Feature vectors may be used to generate clusters representing the user's behaviors over time, and the clusters (e.g., the most prominent k clusters, e.g., k=3) may be used to represent the user's baselines. Clustering may be of any suitable type, such a GMM, K-mean, etc. These clusters may be updated over time as additional user log data is generated. In particular embodiments, the user's baseline data may be compared to other user baseline data, e.g., baseline data of other users who share similar characteristics (e.g., age) as the user. In particular embodiments, the user's baseline data may be compared to the user's behavior over a period of time (e.g., an hour, a day, etc.). Deviations between a user's baseline and other users' baselines, or deviations between a user's recent or current behavior and the user's own baseline, or both, may be used to detect abnormalities in the user's behavior, possibly indicating that the user requires assistance or prompting the system or another connected device to check on the user's welfare. In particular embodiments, an activity state pattern is learned over time by updating the cluster centroids for the user until convergence is obtained beyond a certain threshold. In particular embodiments, when a user's centroids don't change beyond that threshold over a certain amount of time (e.g., a number of days), then those centroids may be used to represent the user's baseline.

In particular embodiments, an orientation of a sensor, such as an IMU, may vary between real-world uses, for example from user to user or from one particular instance of a device to another particular instance (e.g., due to variation in device manufacturing). For example, the orientation of an IMU unit in a set of earbuds could vary from one device/user combination to another device/user combination. These differences can affect the accuracy of the data collected. In particular embodiments, an initial device calibration phase may be used to improve the system's accuracy. For example, a calibration phase may be initiated during the first use of a device, or during the first use of a device by a particular user (e.g., the resulting calibration may be both device and user specific). In particular embodiments, a calibration phase may include a particular duration (e.g., 30 seconds) during which continuous recoding of sensor data occurs. For example, collected IMU sensor data may be smoothed, a 10-axis UMU baseline calculation may be performed, and the an axis transfer function is calculated.

A calibration phase may include instructions to a user to put the device in a particular position and/or for the user to adopt a particular pose. For example, for a pair of earbuds a user may be instructed to wear the earbuds while standing still with their head upright, in a normal standing position. The generated transfer function may be saved and subsequently used by the system to convert IMU signals to the axis system in which the activity-logging systems were trained.

This disclosure contemplates that sensor data from any suitable number of devices may be used to classify user activity. For example, in addition to a wrist-worn device and a head-worn device, smartphones contain motion sensors and could be utilized by a system for activity state recognition. For example, the location or use of the smartphone may be identified, and if the location or use corresponds to reliable data collection regarding the user's activity location (e.g., the phone is not merely sitting on a table or placed in a backpack or purse), then data from the smartphone may be used as an additional signal for determining user activity.

FIG. 6 illustrates an example computer system 600. In particular embodiments, one or more computer systems 600 perform one or more steps of one or more methods described or illustrated herein. In particular embodiments, one or more computer systems 600 provide functionality described or illustrated herein. In particular embodiments, software running on one or more computer systems 600 performs one or more steps of one or more methods described or illustrated herein or provides functionality described or illustrated herein. Particular embodiments include one or more portions of one or more computer systems 600. Herein, reference to a computer system may encompass a computing device, and vice versa, where appropriate. Moreover, reference to a computer system may encompass one or more computer systems, where appropriate.

This disclosure contemplates any suitable number of computer systems 600. This disclosure contemplates computer system 600 taking any suitable physical form. As example and not by way of limitation, computer system 600 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement.

In particular embodiments, processor 602 includes hardware for executing instructions, such as those making up a computer program. As an example and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In particular embodiments, processor 602 may include one or more internal caches for data, instructions, or addresses. This disclosure contemplates processor 602 including any suitable number of any suitable internal caches, where appropriate. As an example and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602. Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In particular embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In particular embodiments, memory 604 includes main memory for storing instructions for processor 602 to execute or data for processor 602 to operate on. As an example and not by way of limitation, computer system 600 may load instructions from storage 606 or another source (such as, for example, another computer system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In particular embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In particular embodiments, one or more memory management units (MMUs) reside between processor 602 and memory 604 and facilitate accesses to memory 604 requested by processor 602. In particular embodiments, memory 604 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory, this disclosure contemplates any suitable memory.

In particular embodiments, storage 606 includes mass storage for data or instructions. As an example and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computer system 600, where appropriate. In particular embodiments, storage 606 is non-volatile, solid-state memory. In particular embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In particular embodiments, I/O interface 608 includes hardware, software, or both, providing one or more interfaces for communication between computer system 600 and one or more I/O devices. Computer system 600 may include one or more of these I/O devices, where appropriate. One or more of these I/O devices may enable communication between a person and computer system 600. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In particular embodiments, communication interface 610 includes hardware, software, or both providing one or more interfaces for communication (such as, for example, packet-based communication) between computer system 600 and one or more other computer systems 600 or one or more networks. As an example and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it. As an example and not by way of limitation, computer system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In particular embodiments, bus 612 includes hardware, software, or both coupling components of computer system 600 to each other. As an example and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing first sensor data from a first sensor worn on a first portion of a user's body;
accessing second sensor data from a second sensor worn on a second portion of the user's body;
determining, based on both the first sensor data and the second sensor data, one or more first features related to the user's activity;
determining, based on the one or more first features, an initial classification of the user's activity;
determining whether the initial classification indicates a class that includes one or more subclasses that are more distinguishable by one of the sensors relative to the other sensor; and
when the initial classification indicates a class that includes one or more subclasses that are more distinguishable by one of the sensors relative to the other sensor, then:
determining one or more second features based solely on sensor data from the one sensor; and
determining, based on the second features, a specific subclassification of the user's activity; or
when the initial classification does not indicate a class that includes one or more subclasses that are more distinguishable by one of the sensors relative to the other sensor, then determining a final classification of the user's activity based on the one or more first features.

2. The method of claim 1, wherein the first portion of a user's body comprises a limb and the second portion of the user's body comprises a trunk or a head.

3. The method of claim 2, wherein the first portion of the user's body comprise an arm and the second portion of the user's body comprises the head.

4. The method of claim 3, wherein the first sensor is part of a wrist-worn device.

5. The method of claim 4, wherein the wrist-worn device comprises a watch and the second sensor comprises a pair of earbuds.

6. The method of claim 3, wherein the initial classification is one classification from a set of classifications comprising: user motion, user non-motion, and user posture.

7. The method of claim 6, wherein the user posture classification indicates a class that includes a set of subclasses comprising: standing, sitting, and lying down.

8. The method of claim 7, wherein:
the first sensor is more able to distinguish between the standing and sitting subclasses than is the second sensor; and
the second sensor is more able to distinguish between the sitting and lying down subclasses than is the first sensor.

9. The method of claim 1, further comprising:
accessing a stream of classified user-activity data; and
changing a classification of at least a portion of the classified user-activity data based on one or more characteristics of the stream of classified user-activity data.

10. The method of claim 9, wherein changing a classification of at least a portion of the stream of classified user-activity data based on one or more characteristics of the stream of classified user-activity data comprising changing a classification of the portion of the stream of classified user-activity data when that portion is less than a threshold size.

11. The method of claim 1, further comprising:
determining that a segment of the first sensor data or a segment of the second sensor data is unreliable, based on a comparison of the segment with a threshold or with a data signature indicating how a device that includes the sensor generating the segment is worn on the user's body; and
in response to the determination that the segment of sensor data is unreliable, then excluding that segment from sensor data that is accessed.

12. The method of claim 1, further comprising:
accessing information identifying a context associated with the user during a time associated with the first sensor data and the second sensor data; and
determining, based on the set of features and on a set of class weights associated with the identified context, the initial classification of the user's activity.

13. The method of claim 1, wherein either or both of the first sensor and the second sensor are initially calibrated to the user.

14. The method of claim 1, further comprising adding to a user activity log an identification of the specific subclassification or of the final classification and a length of time associated with that subclassification or with that final classification.

15. One or more non-transitory computer readable storage media embodying instructions and coupled to one or more processors that are operable to execute the instructions to:
access first sensor data from a first sensor worn on a first portion of a user's body;
access second sensor data from a second sensor worn on a second portion of the user's body;
determine, based on both the first sensor data and the second sensor data, one or more first features related to the user's activity;
determine, based on the one or more first features, an initial classification of the user's activity;
determine whether the initial classification indicates a class that includes one or more subclasses that are more distinguishable by one of the sensors relative to the other sensor; and
when the initial classification indicates a class that includes one or more subclasses that are more distinguishable by one of the sensors relative to the other sensor, then:
determine one or more second features based solely on sensor data from the one sensor; and
determine, based on the second features, a specific subclassification of the user's activity; or
when the initial classification does not indicate a class that includes one or more subclasses that are more distinguishable by one of the sensors relative to the other sensor, then determine a final classification of the user's activity based on the one or more first features.

16. The media of claim 15, wherein the first portion of a user's body comprises a limb and the second portion of the user's body comprises a trunk or a head.

17. The media of claim 16, wherein the first portion of the user's body comprise an arm and the second portion of the user's body comprises the head.

18. A system comprising:
one or more non-transitory computer readable storage media embodying instructions; and
one or more processors coupled to the non-transitory computer readable storage media, the one or more processors being operable to execute the instructions to:
access first sensor data from a first sensor worn on a first portion of a user's body;
access second sensor data from a second sensor worn on a second portion of the user's body;
determine, based on both the first sensor data and the second sensor data, one or more first features related to the user's activity;
determine, based on the one or more first features, an initial classification of the user's activity;
determine whether the initial classification indicates a class that includes one or more subclasses that are more distinguishable by one of the sensors relative to the other sensor; and
when the initial classification indicates a class that includes one or more subclasses that are more distinguishable by one of the sensors relative to the other sensor, then:
determine one or more second features based solely on sensor data from the one sensor; and
determine, based on the second features, a specific subclassification of the user's activity; or
when the initial classification does not indicate a class that includes one or more subclasses that are more distinguishable by one of the sensors relative to the other sensor, then determine a final classification of the user's activity based on the one or more first features.

19. The system of claim 18, wherein the first portion of a user's body comprises a limb and the second portion of the user's body comprises a trunk or a head.

20. The system of claim 19, wherein the first portion of the user's body comprise an arm and the second portion of the user's body comprises the head.
